(19) United States
(12) Patent Application Publication
(10) Pub. No.: US 2006/0270631 A1
(43) Pub. Date: Nov. 30, 2006

(54) Methods for the treatment and prevention of angiogenic diseases

(76) Inventors: Charles D. Smith, Isle of Palms, SC (US); Kevin J. French, Harrisburg, PA (US); Lynn W. Maines, Hummelstown, PA (US)

Correspondence Address: Charles D. Smith, 280 Calhoun Street, Box 250140, MUSC, Charleston, PA, 29425, US

(21) Appl. No.: 11/439,672

(22) Filed: May 24, 2006

Related U.S. Application Data
(60) Provisional application No. 60/684,761, filed on May 26, 2005.

(51) Int. Cl. A61K 31/685

(52) U.S. Cl. 514/78

(57) ABSTRACT

The invention includes processes mainly for the treatment of angiogenic diseases, such as diabetic retinopathy, arthritis, cancer, psoriasis, Kaposi's sarcoma, hemangiomas, myocardial angiogenesis, atherosclerosis, and ocular angiogenic diseases such as choroidal neovascularization, retinopathy of prematurity (retrolental fibroplasias), macular degeneration, corneal graft rejection, rubeosis, neuroscular glacoma and Oster Webber syndrome. The processes involve treating a patient with a pharmaceutical composition containing an active ingredient that inhibits the activity of sphingosine kinase.

### REFERENCE TO RELATED APPLICATIONS

This application is a non-provisional application claiming priority under 35 U.S.C. section 119(e) to provisional application No. 60/684,761 filed May 26, 2005, the contents of which are incorporated herein by reference.

### GOVERNMENT SPONSORSHIP

This invention was made with government support Grant EY016608 awarded by the United States Public Health Service. Accordingly, the US government may have certain rights in this invention.

### FIELD OF THE INVENTION

The present invention relates to methods useful for the treatment and/or prevention of diseases that involve undesired angiogenesis. More specifically, the invention relates to the use of chemical compounds and compositions that inhibit the enzymatic activity of sphingosine kinase for the treatment and/or prevention of angiogenic diseases, such as diabetic retinopathy, arthritis, cancer, psoriasis, Kaposi's sarcoma, hemangiomas, myocardial angiogenesis, atherosclerosis, and ocular angiogenic diseases, such as choroidal neovascularization, retinopathy of prematurity (retrolental fibroplasias), macular degeneration, corneal graft rejection, rubeosis, neuroscular glacoma and Oster Webber syndrome.

### BACKGROUND OF THE INVENTION

A. The role of sphingosine kinase (SK) in angiogenic diseases.

Angiogenesis refers to the state in the body in which various growth factors or other stimuli promote the formation of new blood vessels. As discussed below, this process is critical to the pathology of a variety of diseases. In each case, excessive angiogenesis allows the progression of the disease and/or the produces undesired effects in the patient. Since conserved biochemical mechanisms regulate the proliferation of vascular endothelial cells that form these new blood vessels, i.e. neovascularization, identification of methods to inhibit these mechanisms are expected to have utility for the treatment and/or prevention of a variety of diseases. The following discussion provides further details in how the methods of the present invention can be used to inhibit angiogenesis in several of these diseases.

The mechanisms and effects of the sphingolipid interconversion have been the subjects of a growing body of scientific investigation. Sphingomyelin is not only a structural component of cellular membranes, but also serves as the precursor for the potent bioactive lipids ceramide and sphingosine 1-phosphate (S1P). A ceramide:S1P rheostat is thought to determine the fate of the cell, such that the relative cellular concentrations of ceramide and S1P determine whether a cell proliferates or undergoes apoptosis. Ceramide is produced by the hydrolysis of sphingomyelin in response to angiogenic growth factors, such as Vascular Endothelial Growth Factor (VEGF), and inflammatory stresses, including Tumor Necrosis Factor-α (TNFα), and can be hydrolyzed by ceramidase to produce sphingosine. Sphingosine is then rapidly phosphorylated by sphingosine kinase (SK) to produce S1P. Ceramidase and SK are also activated by cytokines and growth factors, leading to rapid increases in the intracellular levels of S1P and depletion of ceramide levels. This situation promotes cell proliferation and inhibits apoptosis, and is a key process in angiogenesis and inflammation.

1. Diabetic Retinopathy.

Diabetic retinopathy is a leading cause of vision impairment (Gardner, Trans Am Ophthalmol Soc 93: 583 (1995)), and elevation in the expression of growth factors contributes to pathogenic angiogenesis in this disease. In particular, vascular endothelial growth factor (VEGF) is a prominent contributor to the new vessel formation in the diabetic retina (Frank et al., Arch Ophthalmol 115: 1036 (1997), Sone et al., Diabetologia 40: 726 (1997)), and VEGF has been shown to be elevated in patients with proliferative diabetic retinopathy (Aiello et al., N Engl J Med 331: 1480 (1994)). In addition to diabetic retinopathy, several other debilitating ocular diseases, including age-related macular degeneration and choroidal neovascularization, are associated with excessive angiogenesis that is mediated by VEGF and other growth factors (Grant et al., Expert Opin Investig Drugs 13: 1275 (2004), Campochiaro, Expert Opin Biol Ther 4: 1395 (2004), Schlingemann, Graefes Arch Clin Exp Ophthalmol 242: 91 (2004), Das et al., Prog Retin Eye Res 22: 721 (2003), Adamis et al., Angiogenesis 3: 9 (1999)).

In the retina, VEGF is expressed in the pigmented epithelium, the neurosensory retina, the pericytes and the vascular smooth muscle layer (Murata et al., Lab Invest 74: 819 (1996), Hammes et al., Diabetes 47: 401 (1998)). VEGF induces endothelial cell proliferation, favoring the formation of new vessels in the retina (Pe'er et al., Lab Invest 72: 638 (1995)). At the same time, basic fibroblast growth factor (bFGF) in the retina is activated, and this factor acts in synergy with VEGF such that the two together induce the formation of new vessels in which the subendothelial matrix is much weaker than in normal vessels (Jonca et al., J Biol Chem 272: 24203 (1997), Sahara, Circulation 92: 365 (1995)). Additionally, VEGF facilitates fluid extravasation in the interstitium, where exudates form in the retinal tissue (Murata et al., Lab Invest 74: 819 (1996), Hammes et al., Diabetes 47: 401 (1998)). VEGF also promotes the fenestration of endothelial cells, a process that can give rise to intercellular channels through which fluids can leak (Roberts et al., J Cell Sci 108 (Pt 6): 2369 (1995)), and disrupts tight junctions between cells (Antonetti et al., J Biol Chem 274: 23463 (1999)). Thus, reduction of VEGF activity in the retina is likely to efficiently reduce the development and progression of retinal angiogenesis and vascular leakage which underlie the retinopathic process.

The pro-inflammatory cytokine TNFα has also been demonstrated to play a role in diabetic retinopathy since it alters the cytoskeleton of endothelial cells, resulting in leaky barrier function and endothelial cell activation (Camussi et al., Int Arch Allergy Appl Immunol 96: 84 (1991)). These changes in retinal endothelial cells are central in the pathologies of diabetic retinopathy.

A link between the actions of VEGF and sphingosine kinase (SK) may be involved in driving retinopathy. SK has been shown to mediate VEGF-induced activation of ras- and mitogen-activated protein kinases (Shu et al., Mol Cell Biol 22: 7758 (2002)). VEGF has been shown to enhance intracellular signaling responses to S1P, thereby increasing its angiogenic actions (Igarashi et al., Proc Natl Acad Sci USA 100: 10664 (2003)). S1P has also been shown to stimulate NFκB activity (Xia et al., Proc Natl Acad Sci USA 95: 14196 (1998)) leading to the production of COX-2, adhesion molecules and additional VEGF production, all of which have been linked to angiogenesis (Yeh et al., Invest Ophthalmol Vis Sci 45: 2368 (2004), Guastalla et al., Bull Cancer 91 Spec No: S99 (2004)). Furthermore, the expression of endothelial isoforms of nitric oxide synthase (eNOS) is regulated by SK (Igarashi et al., J Biol Chem 275: 32363 (2000), Igarashi et al., J Biol Chem 276: 12420 (2001), Igarashi et al., J Biol Chem 276: 36281 (2001)), and eNOS too subsequently modulates angiogenesis (Rikitake et al., Arterioscler Thromb Vasc Biol 22: 108 (2002)). Clearly, SK is a central regulator of angiogenesis, supporting our hypothesis that its pharmacological manipulation may be therapeutically useful.

One of the most attractive sites of intervention in this pathway is the conversion of sphingosine to S1P by the enzyme sphingosine kinase (SK). SK is the key enzyme responsible for the production of S1P synthesis in mammalian cells, which facilitates cell survival and proliferation (Cuvillier, Biochim Biophys Acta 1585: 153 (2002)), and mediates critical processes involved in angiogenesis and inflammation, including responses to VEGF (Shu et al., Mol Cell Biol 22: 7758 (2002)) and TNFα (Xia et al., Proc Natl Acad Sci USA 95: 14196 (1998), Chen et al., Am J Physiol Heart Circ Physiol 287: H1452 (2004)). Therefore, inhibition of S1P production is a potentially important point of therapeutic intervention for diabetic retinopathy.

2. Arthritis

Rheumatoid arthritis (RA) is a chronic, systemic disease that is characterized by synovial hyperplasia, massive cellular infiltration, erosion of the cartilage and bone, and an abnormal immune response (Kohl et al., Nat Med 1: 792 (1995)). Studies on the etiology and therapy of rheumatoid arthritis have been greatly facilitated by the development of animal models that mimic the clinical and immunopathological disorders seen in humans. From studies in these models, it is clear that the full manifestations of RA are dependent on interactions between immune cells, endothelial cells and specialized cells in the joints, including chondrocytes and synoviocytes. This includes angiogenic and inflammatory processes.

The early phase of rheumatic inflammation is characterized by leukocyte infiltration into tissues, especially by neutrophils. In the case of RA, this occurs primarily in joints where leukocyte infiltration results in synovitis and synovium thickening producing the typical symptoms of warmth, redness, swelling and pain. As the disease progresses, the aberrant collection of cells invade and destroy the cartilage and bone within the joint leading to deformities and chronic pain. The inflammatory cytokines TNFα, IL-1β and IL-8 act as critical mediators of this infiltration, and these cytokines are present in the synovial fluid of patients with RA.

Leukocytes localize to sites of inflammatory injury as a result of the integrated actions of adhesion molecules, cytokines, and chemotactic factors. The adherence of neutrophils to the vascular endothelium is a first step in the extravasation of cells into the interstitium. This process is mediated by selectins, integrins, and endothelial adhesion molecules, e.g. ICAM-1 and VCAM-1. Since TNFα induces the expression of ICAM-1 and VCAM-1 and is present in high concentrations in arthritic joints, it is likely that this protein plays a central role in the pathogenesis of the disease. A further critical process in the progression of RA is the enhancement of the blood supply to the synovium through angiogenesis. Expression of the key angiogenic factor VEGF is potently induced by pro-inflammatory cytokines including TNFα (Taylor, Arthritis Res 4 (Suppl 3): S99 (2002)). Together, these data point to important roles of TNFα, leukocytes, leukocyte adhesion molecules, leukocyte chemoattractants and angiogenesis in the pathogenesis of arthritic injury.

Early in the disease, immunologic reactions or other activating signals promote the release of inflammatory cytokines, particularly TNFα and IL-1β from macrophages and mast cells. Ceramide is produced by the hydrolysis of sphingomyelin in response to inflammatory stresses, including TNFα and IL-1β (Dressler et al., Science 255: 1715 (1992)). Ceramide can be further hydrolyzed by ceramidase to produce sphingosine which is then rapidly phosphorylated by SK to produce S1P. In addition to its role in regulating cell proliferation and apoptosis, S1P is a central player in the pathway since it has pleiotropic actions on the endothelial cells, leukocytes, chondrocytes and synovial cells. Within the endothelial cells, S1P activates NFκB thereby inducing the expression of multiple adhesion molecules and COX-2 resulting in PGE₂ synthesis. Together, this chemoattractant and the adhesion molecules promote neutrophil infiltration into the synovium. At the same time, S1P directly activates the neutrophils resulting in the release of oxygen free radicals that destroy joint tissue (Perez-Simon et al., Blood 100: 3121 (2002)). Progression of RA is associated with a change from a Th1 to a Th2 environment, and sphingosine is selectively inhibitory toward Th1 cells. Consequently, inhibiting the conversion of sphingosine to S1P should attenuate the progression of the disease. Platelets, monocytes and mast cells secrete S1P upon activation, promoting inflammatory cascades at the site of tissue damage (Yatomi et al., Blood 86: 193 (1995)). S1P also promotes the secretion of proteases from chondrocytes that contribute to joint destruction. Finally, S1P-mediated expression of VEGF promotes the angiogenesis necessary to support the hyperproliferation of synovial cells.

According to this model, two major targets for new anti-RA therapies can be defined: TNFα and S1P. The use of inhibitors of SK as anti-RA agents has not been previously demonstrated. The following Examples demonstrate that SK inhibitors block S1P production in endothelial cells and prevent their activation by VEGF, making these compounds useful for the treatment and/or prevention of RA.

3. Cancer

The role of angiogenesis in cancer is well recognized (Baluk et al., Curr Opin Genet Dev 15: 102 (2005), Collins et al., Semin Oncol 32: 61 (2005), Dhanabal et al., Curr Med Chem Anti-Canc Agents 5: 115 (2005), Ferrara, Exs 209 (2005), Gasparini et al., J Clin Oncol 23: 1295 (2005), Hicklin et al., J Clin Oncol 23: 1011 (2005), Podar et al., Blood 105: 1383 (2005), Ribatti, Br J Haematol 128: 303 (2005), Schneideret al., J Clin Oncol 23: 1782 (2005)). Growth of a tumor is absolutely dependent on neovascularization so that nutrients can be provided to the tumor cells. The major factor that promotes endothelial cell proliferation during tumor neovascularization is VEGF. As discussed above, signaling through VEGF receptors is dependent on the actions of SK, and the newly discovered SK inhibitors are shown in the Examples that follow to inhibit endothelial cell responses to VEGF. Furthermore, data demonstrating that the SK inhibitors have anti-tumor activity in vivo provide further evidence that inhibition of angiogenesis by these compounds has antitumor activity. Therefore, the methods of this invention will have utility for the treatment of cancer.

4. Atherosclerosis

Angiogenesis contributes to atherosclerosis, a major cause of death of Western populations. Atherosclerosis is the main cause of heart attack. The walls of the coronary artery are normally free of microvessels except in the atherosclerotic plaques, where there are dense networks of capillaries, known as the vasa vasorum. These fragile microvessels can cause hemorrhages, leading to blood clotting, with a subsequent decreased blood flow to the heart muscle and heart attack. Atherosclerosis is a complex vascular disease that involves a series of coordinated cellular and molecular events characteristic of angiogenic and inflammatory reactions. In response to vascular injury, the first atherosclerotic lesions are initiated by acute inflammatory reactions, mostly mediated by monocytes, platelets and T lymphocytes. These inflammatory cells are activated and recruited into the subendothelial vascular space through locally expressed chemotactic factors and adhesion molecules expressed on endothelial cell surface. Continuous recruitment of additional circulating inflammatory cells into the injured vascular wall potentiates the inflammatory reaction by further activating vascular smooth muscle (VSM) cell migration and proliferation. This chronic vascular inflammatory reaction leads to fibrous cap formation, which is an oxidant-rich inflammatory milieu composed of monocytes/macrophages and VSM cells. Over time, this fibrous cap can be destabilized and ruptured by extracellular metalloproteinases secreted by resident monocytes/ macrophages. The ruptured fibrous cap can easily occlude vessels resulting in acute cardiac or cerebral ischemia. This underlying mechanism of atherosclerosis indicates that activation of monocyte/macrophage and VSM cell migration and proliferation play critical roles in the development and progression of atherosclerotic lesions. Importantly, it also suggests that a therapeutic approach that smooth muscle cell proliferation should be able to prevent the progression and/or development of atherosclerosis.

SK is highly expressed in platelets allowing them to phosphorylate circulating sphingosine to produce S1P. In response to vessel injury, platelets release large amounts of S1P into the sites of injury which can exert mitogenic effects on VSM cells by activating S1P receptors. S1P is also produced in activated endothelial and VSM cells. In these cells, intracellularly produced S1P functions as a second messenger molecule, regulating Ca²⁺ homeostasis associated with cell proliferation and suppression of apoptosis. Additionally, deregulation of apoptosis in phagocytes is an important component of the chronic inflammatory state of atherosclerosis, and S1P protects granulocytes from apoptosis. Together, these studies indicate that activation of SK alters sphingolipid metabolism in favor of S1P formation, resulting in pro-inflammatory and hyper-proliferative cellular responses.

According to this model, SK is a major target for new anti-atherosclerosis therapies. The use of inhibitors of SK as anti-atherosclerosis agents has not been previously demonstrated. The following Examples demonstrate that SK inhibitors prevent VEGF activation of endothelial cells. This will prevent the deleterious activation of leukocytes, as well as prevent infiltration and smooth muscle cell hyperproliferation, making these compounds useful for the treatment and/or prevention of atherosclerosis.

5. Other angiogenic diseases

Choroidal Neovascularization. More than 50 eye diseases have been linked to the formation of choroidal neovascularization, although the three main diseases that cause this pathology are age-related macular degeneration, myopia and ocular trauma. Even though most of these causes are idiopathic, among the known causes are related to degeneration, infections, choroidal tumors and or trauma. Among soft contact lens wearers, choroidal neovascularization can be caused by the lack of oxygen to the eyeball.

Hemangiomas are angiogenic diseases characterized by the proliferation of capillary endothelium with accumulation of mast cells, fibroblasts and macrophages. They represent the most frequent tumors of infancy, and are characterized by rapid neonatal growth (proliferating phase). By the age of 6 to 10 months, the hemangioma's growth rate becomes proportional to the growth rate of the child, followed by a very slow regression for the next 5 to 8 years (involuting phase). Most hemangiomas occur as single tumors, whereas about 20% of the affected infants have multiple tumors, which may appear at any body site. Several studies have provided insight into the histopathology of these lesions. In particular, proliferating hemangiomas express high levels of proliferating cell nuclear antigen (a marker for cells in the S phase), type IV collagenase, VEGF and FGF-2.

Psoriasis and Kaposi's sarcoma are angiogenic and proliferative disorders of the skin. Hypervascular psoriatic lesions express high levels of the angiogenic inducer IL-8, whereas the expression of the endogenous inhibitor TSP-1 is decreased. Kaposi's sarcoma (KS) is the most common tumor associated with human immunodeficiency virus (HIV) infection and is in this setting almost always associated with infection by human herpes virus 8. Typical features of KS are proliferating spindle-shaped cells, considered to be the tumor cells and endothelial cells forming blood vessels. KS is a cytokine-mediated disease, highly responsive to different inflammatory mediators like IL-1β, TNF-α and IFN-γ and angiogenic factors.

B. Sphingosine kinase enzymology and pharmacology.

Sphingosine kinase catalyzes the production of S1P in cells. RNA encoding SK is detected in most tissues, with higher levels in lung and spleen. A number of studies have shown that a variety of proliferative factors, including PKC activators, fetal calf serum and platelet-derived growth factor, EGF, and TNFα (Xia et al., Proc Natl Acad Sci USA 95: 14196 (1998)) rapidly elevate cellular SK activity.

In spite of the high level of interest in sphingolipid-mediated signaling, there are very few known inhibitors of the enzymes of this pathway. Pharmacological studies to date have used three compounds to inhibit SK activity: dimethylsphingosine (DMS), D,L-threo-dihydrosphingosine and N,N,N-trimethylsphingosine. However, these compounds are not specific inhibitors of SK and have been shown to affect protein kinase C (Igarashi et al., Biochemistry 28: 6796 (1989)), sphingosine-dependent protein kinase (Megidish et al., Biochem Biophys Res Commun 216: 739 (1995)), 3-phosphoinositide-dependent kinase (King et al., J Biol Chem 275: 18108 (2000)), and casein kinase II (McDonald et al., J Biol Chem 266: 21773 (1991)). Therefore, improved inhibitors of SK are required not only for basic research, but also as lead compounds for developing novel drugs. To this end, a series of structurally novel inhibitors of SK was identified (French et al., Cancer Res 63: 5962 (2003)). These compounds inhibit endogenous S1P formation in intact cancer cells while inducing apoptosis, and demonstrate a high degree of selectivity for SK versus other lipid and protein kinases. We have developed additional SK inhibitors that have activity in both cell and animal models. As demonstrated in the following Examples, these SK inhibitors can be chronically administered without systemic toxicity. Because of their excellent pharmacological properties, these new SK inhibitors provide agents for the practice of therapies that inhibit SK activity in target cells within an animal.

### SUMMARY OF THE INVENTION

The invention is methods for the use of compounds and pharmaceutical compositions for the treatment of angiogenic diseases. The compounds, and the active ingredient of the compositions, inhibit the activity of human sphingosine kinase (SK).

### DESCRIPTION OF THE DRAWINGS

FIG. 1. SK inhibitors. Representative compounds with SK inhibitory activity are shown.

FIG. 2. Inhibition of S1P production in HUVECs by SK inhibitors. Human endothelial cells were incubated with the indicated concentration of Compound I (▾), Compound II (♦), Compound V (●), ABC294640 (▪) or ABC747080 (▴) before the addition of 0.4 μCi of [³H]sphingosine. After 15 minutes, cells were lysed and extracted with chloroform: methanol, and the amounts of [³H]sphingosine in the organic phase and [³H]S1P in the aqueous phase were then determined. Values represent the mean±sd SK activity.

FIG. 3. Inhibition of cellular SK activity in RECs. Bovine retinal endothelial cells were incubated with 20 μM dimethylsphingosine (DMS) or 25 μg/mL Compound I, Compound II or Compound V (approximately 80 μM for each compound) for 4 hours before the addition of 0.4 μCi of [³H]sphingosine. After 15 minutes, cells were lysed and extracted with chloroform: methanol. The total amounts of [³H]sphingosine in the organic phase and [³H]S1P in the aqueous phase were then determined. Values represent the mean±SD of duplicate samples in a typical experiment.

FIG. 4. Toxicity of Compound II toward HRECs. Human RECs were serum-starved for 24 hours and then left untreated for 12 hours (▪) or incubated with 50 ng/mL VEGF (▴) for 14 hours in the presence of the indicated concentration of Compound II. These protocols were chosen to exactly match conditions in SK activity and proliferation experiments described below. After incubation, the percentages of cells that survived the treatment were determined. Values represent the mean i SD cell survival in triplicate samples in a typical experiment.

FIG. 5. Effects of Compound II on VEGF-stimulated SK activity in RECs. Bovine RECs were serum-starved for 24 hours and then left untreated or incubated with VEGF (50 ng/mL) in the presence of DMS (D) or the indicated concentration (in μM) of Compound II for 12 hours. [³H]Sphingosine was then added to the cells and its conversion to [³H]S1P was determined. Values represent the mean±SD of duplicate samples in a typical experiment.

FIG. 6. Effects of Compound II on VEGF-stimulated proliferation of RECs. Human RECs were serum-starved for 24 hours and then left untreated or incubated with VEGF (50 ng/mL) in the presence of the indicated concentration (in μM) of Compound II for 12 hours. [3H]Thymidine was then added and the cultures were incubated an additional 2 hours. The amount of [³H]thymidine incorporated into DNA was determined. Values represent the mean±SD of duplicate samples in a typical experiment.

FIG. 7. Effects of SK inhibitors on VEGF-induced vascular leakage. Nude mice were treated with either DMSO (as the solvent control) or an SK inhibitor. After 30 minutes, Evan's Blue dye was injected intravenously and the animals received subsequent subcutaneous injects of either PBS or 400 ng of VEGF. Panel A. The areas of vascular leakage in each animal was quantified (n=3 and 5 for control and Compound II-treated animals, respectively). Panel B. Nude mice were injected intraperitoneally with carrier (Control, open bar) or 75 mg/kg ABC294640 (hatched bar) or given 100 mg/kg ABC294640 by oral gavage (solid bar), followed by administration of Evan's Blue dye and VEGF as indicated above. Values represent the mean±SD areas of vascular leakage. *p<0.01.

FIG. 8. Increased retinal vascular permeability in diabetic rats. Sprague-Dawley rats were injected with buffer (Control) or streptozotocin (Diabetic) and left untreated for 45 days. At that time, animals were injected intravenously with FITC-BSA, and after 30 minutes the animals were sacrificed. Each retina was harvested, sectioned and imaged by fluorescence microscopy. The relative light intensity of FITC-BSA in the inner plexiform and outer nuclear layers from Control (open bars) and Diabetic (shaded bars) rats were quantified. Values represent the mean±sd for 4 rats.

FIG. 9. Effects of ABC 294640 on retinal vascular permeability in diabetic rats. From Day 45 through Day 87, Control (open bars) and Diabetic rats were treated with solvent (shaded bars) or ABC294640 at 25 mg/kg (horizontal-hatched bars) or 75 mg/kg (cross-hatched bars). On Day 87, retinal leakage in each animal was measured. Values represent the mean±sd for 3-5 rats per group.

FIG. 10. Effects of SK inhibitors on disease progression in the CIA model in mice. Female DBA/1 mice were injected with collagen, boosted after 3 weeks and then monitored for symptoms of arthritis. Upon disease manifestation, groups of mice were treated for 12 days as follows: (▴) ABC294640 (100 mg/kg given orally each day for 6 days per week); (Δ) ABC747080 (50 mg/kg given orally each day for 6 days per week); or (▪) vehicle (PEG400 given under the same schedule). On the indicated Day of treatment, the average clinical score (▴) and the average hind paw diameter (B) was determined. *p≦0.05 versus PEG400 alone group.

FIG. 11. Effects of ABC294640 on disease progression in the adjuvant-induced arthritis model in rats. Male Lewis rats were injected subcutaneously with Mycobacterium butyricum, and symptoms of immune reactivity were present after 2 weeks. Responsive rats were randomized into treatment groups (n=8 per group), and received oral daily doses of: solvent alone (0.375% Tween-80); 100 mg/kg ABC294640; 35 mg/kg ABC294640; or 5 mg/kg ABC294640, or intraperitoneal injections of indomethacin (5 mg/kg) every other day. The severity of disease in each animal was quantified by measurement of the hind paw thickness. Panel A. Time course of hind paw arthritic response. Panel B. Final day (Day 10) hind paw thickness measurements. Panel C. Change in paw thickness of respective group versus non-arthritic rats (naive) at Day 10. *,p<0.05; ***, p<0.001 versus solvent alone group.

FIG. 12. Oral anti-tumor activity of SK inhibitors. Balb/c female mice were injected subcutaneously with JC cells suspended in PBS. After palpable tumor growth, animals were treated by oral gavage of either 100 μl of PEG400 (control, open squares) or 100 mg/kg of ABC747080 (filled squares) or ABC294770 (circles) on odd days. Whole body weight and tumor volume measurement were performed for up to 18 days. *p<0.05. Inset: Averaged body weights of mice from each group during course of study.

FIG. 13. Dose-response studies of oral antitumor activity of ABC294640 and ABC747080. Balb/c female mice were injected s.c. with JC cells suspended in PBS. After palpable tumor growth, animals were treated by oral gavage of either ABC294640 or ABC747080 at the indicated doses on odd days.

### DESCRIPTION OF THE PREFERRED EMBODIMENTS

The present invention provides methods for the use of compounds and pharmaceutical compositions for the treatment of agiogenic diseases. The chemical compounds therein and pharmaceutical compositions of the present invention may be useful in the therapy of angiogenic diseases, such as diabetic retinopathy, arthritis, cancer, psoriasis, Kaposi's sarcoma, hemangiomas, myocardial angiogenesis, atherosclerosis, and ocular angiogenic diseases such as choroidal neovascularization, retinopathy of prematurity (retrolental fibroplasias), macular degeneration, corneal graft rejection, rubeosis, neuroscular glacoma and Oster Webber syndrome.

The compounds and pharmaceutical compositions to be used in the present invention can be used in various protocols for treating animals, including humans. In one embodiment of the methods of the present invention, SK in target cells or tissues in an animal undergoing chemotherapy is inhibited by administering to the animal a pharmaceutical composition in an amount effective to inhibit SK in the target cells or tissues of the animal.

In a particularly preferred embodiment of the use of the methods of the present invention, the compounds or compositions can be used for treating angiogenesis in a patient requiring such treatment, by administering the compound or composition to a patient in an amount effective to inhibit the activation of endothelial cells of said patient. This method would involve administering to a patient with an angiogenic disease a composition in an amount effective to prevent the actions of growth factors or other stimuli on vascular endothelial cells.

In another particularly preferred embodiment of the use of the methods of the present invention, the compounds or compositions can be used in a method for treating diabetic retinopathy in a patient requiring such treatment, by administering the composition to a patient in an amount effective to inhibit the aberrant activation of retinal endothelial cells. This method would involve administering to the patient a compound or composition in an amount effective to inhibit SK activity in the retinal endothelial cells.

In another particularly preferred embodiment of the use of the methods of the present invention, the compounds or compositions can be used in a method for treating arthritis in a patient requiring such treatment, by administering the composition to a patient in an amount effective to inhibit the aberrant activation of macrophages, mast cells, neutrophils, endothelial cells, chondrocytes and/or synovial cells. For example, these methods can be used for treating a patient with rheumatoid arthritis. This method would involve administering to the patient a compound or composition in an amount effective to inhibit SK activity in macrophages, mast cells, neutrophils, endothelial cells, chondrocytes and/or synovial cells.

In another particularly preferred embodiment of the use of the methods of the present invention, the compounds or compositions can be used in a method for treating cancer in a patient requiring such treatment, by administering the composition to a patient in an amount effective to inhibit the aberrant activation of endothelial cells. This method would involve administering to the patient a compound or composition in an amount effective to inhibit SK activity in endothelial cells in the tumor.

In another particularly preferred embodiment of the use of the methods of the present invention, the compounds or compositions can be used in a method for treating an ocular angiogenic disease in a patient requiring such treatment, by administering the composition to a patient in an amount effective to inhibit the aberrant activation of retinal endothelial cells. For example, these methods can be used for treating a patient with choroidal neovascularization, retinopathy of prematurity (retrolental fibroplasias), macular degeneration, corneal graft rejection, rubeosis, neuroscular glacoma or Oster Webber syndrome. This method would involve administering to the patient a compound or composition in an amount effective to inhibit SK activity in retinal endothelial cells.

In view of the beneficial effect of inhibiting SK, it is anticipated that the methods of the present invention will be useful not only for therapeutic treatment following the onset of disease, but also for the prevention of disease in animals, including humans. The methods described herein will be essentially the same whether the compounds or pharmaceutical compositions are being administered for the treatment or prevention of disease.

While the invention has been particularly shown and described with reference to preferred embodiments thereof, it will be understood by those skilled in the art that various alterations in form and detail may be made therein without departing from the spirit and scope of the invention. In particular, the specific method of use of the SK inhibitory compounds and compositions can vary significantly without departing from the discovered methods. Additionally, methods for the treatment of additional diseases that involve undesired angiogenesis within the patient are considered to be within the scope of the following claims.

The Examples, which follow, are illustrative of specific embodiments of the invention, and various uses thereof. They are set forth for explanatory purposes only, and are not to be taken as limiting the invention.

### EXAMPLE 1

Identification of SK inhibitors.

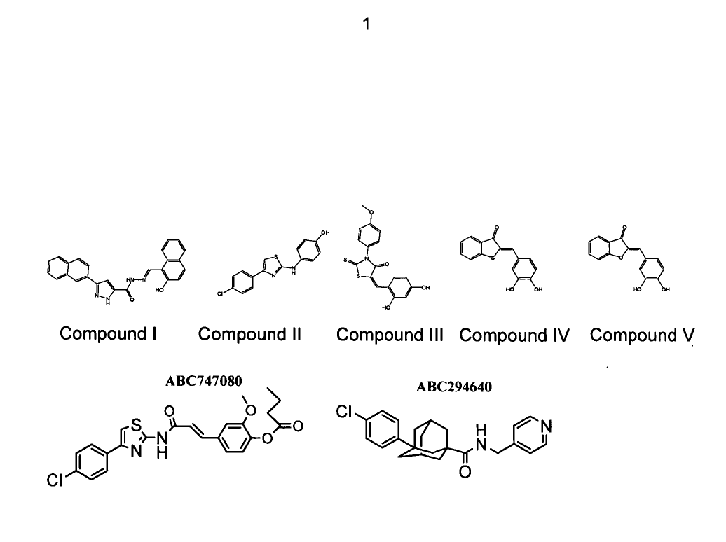

An assay for screening for inhibitors of SK has been established (French et al., Cancer Res 63: 5962 (2003)). A chemical library totaling approximately 16,000 compounds was screened for inhibition of SK. Representative active compounds from four chemotypes of SK inhibitors, designated herein as Compounds I-IV, are shown in FIG. 1. The compounds ABC747080 and ABC294640 (FIG. 1) also inhibit SK.

### EXAMPLE 2

Methods for in vitro studies.

Cell Culture. Primary cultures of bovine retinal endothelial cells (RECs) were isolated as previously described (Maines et al., Neuropharmacology 49: 610 (2005)). Human RECs were purchased from Cell Technologies (catalog number ACBRI181, Kirkland, Wash.) and cultured under identical conditions as those described for bovine RECs. Briefly, the cells were maintained in growth medium consisting of Minimum Essential Medium with D-valine supplemented with 20% fetal calf serum (Gibco, Rockville, Md.), 50 μg/mL of endothelial cell growth supplement (Vec Technologies, Rensslar, N.Y.), 16 U/mL heparin (Fisher Scientific, Pittsburg, Pa.), 0.01 mL/mL MEM vitamins and glutamine (Sigma, St. Louis, Mo.), and 0.02 mL/mL antibiotic/antimycotic (Gibco, Rockville, Md.). The cells were plated on a 25 cm tissue culture flask precoated with fibronectin (Sigma, St. Louis, Mo.) at 2 μg/cm² and were grown in a humidified incubator at 37° C. The medium was removed and fresh medium was added 24 hours following the plating. For experiments on VEGF signaling, the culture medium was replaced with fresh MCDB 131 medium (Sigma, M8537) that lacked fetal calf serum, termed serum-starvation.

Western Blotting. Protein concentrations were determined using the fluorescamine assay (Bohlen et al., Arch Biochem Biophys 155: 213 (1973)) with bovine serum albumin as the standard. Samples were normalized for equal amounts of protein per lane (100 μg), separated by sodium dodecylsulfate-polyacrylamide gel electrophoresis and electrotransferred to nitrocellulose membranes. For SK analyses, membranes were blocked with 5% nonfat milk in Tris-buffered saline with Tween 20 and probed with an anti-SK rabbit polyclonal antibody at a 1:50 dilution, washed and incubated with anti-mouse antibodies conjugated to horseradish peroxidase (1 hr in 3% nonfat milk). The blots were then washed 4 times for 5 minutes at room temperature, developed with SuperSignal development reagents (Pierce Biotechnology, Inc., Rockford, Ill.) and exposed to Kodak XAR film. The following antibodies, along with their appropriate horseradish peroxidase-conjugated secondary antibodies, were used: Erk1/2 (Catalog number 9102, Cell Signaling Technology, Beverly, Mass.), phospho-Erk1/2 (Catalog number 9101, Cell Signaling Technology, Beverly, Mass.), E-selectin (Catalog number 59555, Sigma, St. Louis, Mo.), VCAM-1 (Catalog number CBL206, Chemicon International, Temecula, Calif.), Cox-2 (Catalog number SC-1745, Santa Cruz Biotechnology, Santa Cruz, Calif.) and NFκB (Catalog number 3031, Cell Signaling Technology, Beverly, Mass.).

Cellular S1P formation assay. Cells were grown to confluency in 24-well tissue culture plates and serum-starved for 24 hours as described above. Cells were then treated with 1% DMSO (as the drug vehicle), 20 μM dimethylsphingosine, or the indicated concentration of an SK inhibitor for 4 hours. The cells were then incubated with [³H]sphingosine for 15 minutes, and the formation of [³H]S1P was measured as previously described (French et al., Cancer Res 63: 5962 (2003)). In some assays, the cells were serum-starved for 24 hours and then treated with VEGF (50 ng/mL) alone or in the presence of an SK inhibitor for an additional 12 hours. [³ H]Sphingosine was then added for 15 minutes, and its conversion to [³H]S1P was measured as indicated above.

Cytotoxicity assays. Cells were grown to confluence in 96-well tissue culture plates and serum-starved for 24 hours as described above. Cells were then treated with varying concentrations of Compound II for 12 hours (to parallel the SK activity assays described above) or with varying concentrations of Compound II and 50 ng/mL of VEGF for 14 hours (to parallel the proliferation assays described below). Cell survival was determined using the sulforhodamine assay (Skehan et al., J Natl Cancer Inst 82: 1107 (1990)).

Cell proliferation assay. Cells were grown to confluence in 24-well tissue culture plates and serum-starved overnight as described above. Cells were then treated with 50 ng/mL of VEGF and varying concentrations of Compound II for 12 hours. At that time, 16 μCi of [3H]thymidine was added to each well, and the cultures were incubated for an additional 2 hours. The media was then removed by aspiration, the cells were washed twice with cold PBS, and 0.8 mL of ice-cold 10% trichloroacetic acid was added to each well. After 10 minutes, the trichloroacetic acid was removed by aspiration and replaced with 0.4 mL of 40 μg/mL Type I DNA (Sigma, St. Louis, Mo.) in 0.2 M NaOH. The samples were incubated at 37° C. for 30 minutes, scraped into scintillation vials, and the amount of ³H in the recovered genomic DNA was quantified by scintillation counting.

### EXAMPLE 3

Expression and activity of SK in RECs.

The expression of SK in bovine and human RECs was analyzed by immunoblotting of whole cell lysates using polyclonal antibodies that cross-react with SK from multiple species. Bovine RECs contained high levels of SK protein, exceeding that of endothelial cells from rat brain cortex and JC murine mammary carcinoma cells (ATCC number CRL-2116). Similarly, several preparations of human RECs consistently expressed high amounts of SK, demonstrating that endothelial cells from multiple species express this enzyme.

### EXAMPLE 4

Inhibition of endogenous SK activity by SK inhibitors.

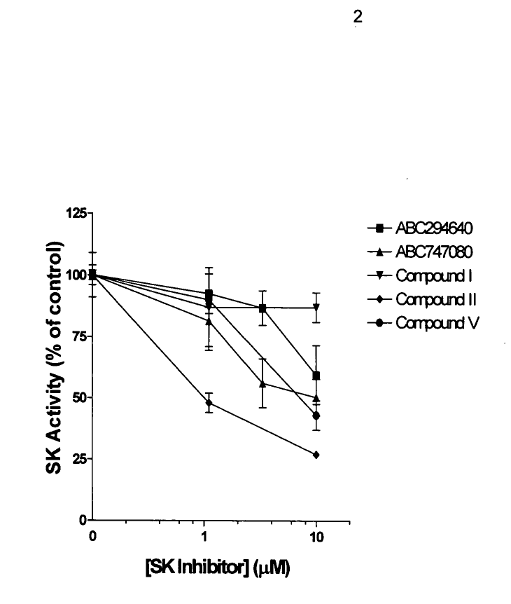

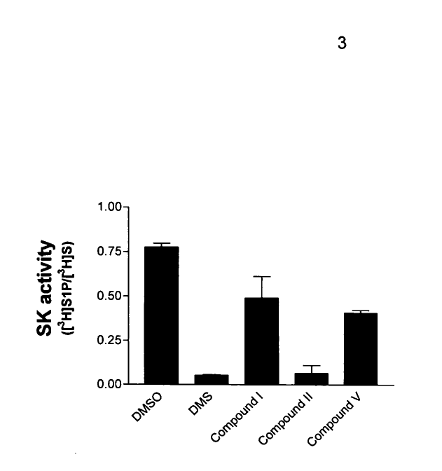

A cell-based assay in which the phosphorylation of exogenously added [³H]sphingosine to [³H]S1P by endogenous SK can be quantified (French et al., Cancer Res 63: 5962 (2003)) was used to evaluate the effects of test compounds on the activity of SK in intact cells. In this assay, cells are incubated with [³ H]sphingosine for an appropriate period of time, and then [³H]sphingosine and [³H]S1P (formed by endogenous SK activity) are separated by extraction and levels of both species are determined by scintillation counting. We have used a number of cell lines in this assay to confirm that the SK inhibitors are active in multiple intact cell systems. For example, human umbilical vein endothelial cells (HUVECs) are commonly used as a model of human vasculature. As demonstrated in FIG. 2, the SK inhibitors cause dose-dependent reductions in the cellular levels of S1P synthesis human umbilical vein endothelial cells. Similarly, the enzymatic activity of SK in bovine RECs, and its sensitivity to SK inhibitors were assessed. FIG. 3 demonstrates the excellent reproducibility of this assay, and the inhibition of SK by the positive control, dimethylsphingosine (DMS). Each of inhibitors of SK (Compound I, Compound II and Compound V) was also active against the endogenous SK activity in the bovine RECs.

In additional experiments summarized in Table 1, the effects of Compound II and ABC747080 on S1P production in multiple cell lines involved in the angiogenic process in arthritis were tested. Specifically, HUVECs, human chondrocytes (HC), and human synovial cells from a patient with RA (HFLS-RA) were examined. Both Compound II and ABC747080 were capable of inhibiting endogenous SK activity in each of these types of cells, with Compound II demonstrating higher potency.

TABLE 1
Compound II and ABC747080 inhibit S1P formation in cells modulating
the inflammatory response in arthritis. Values represent drug
concentrations (in μM) that decrease S1P formation by 50%.

| Test Compound | HUVECs | HCs | HFLS-RA cells |
|---|---|---|---|
| Compound II | 0.2 | 0.36 | 0.19 |
| ABC747080 | 9.2 | 8.5 | 12 |

### EXAMPLE 5

Cytotoxicity of Compound II toward human RECs.

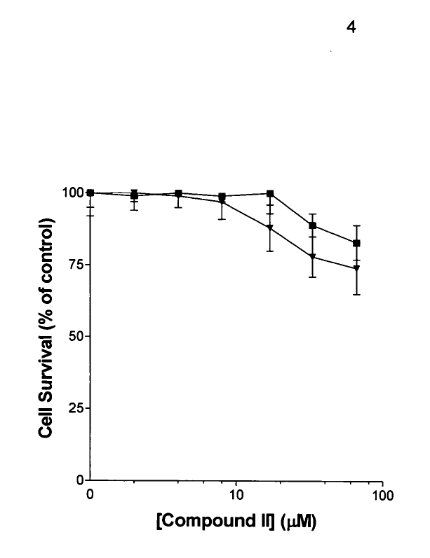

Since Compound II was the most efficacious inhibitor of SK in the RECs, this compound was used to characterize the biological effects of inhibiting SK in these cells. It was first necessary to determine the toxic effects of this compound toward cultures of RECs under conditions identical to subsequent signal studies. Therefore, human RECs were grown to confluence and then incubated in serum-free MCDB 131 medium for 24 hours. These conditions were chosen to mimic the state of endothelial cells in mature retinal microvasculature, and provide cultures that are sensitive to VEGF-induced proliferation (described below). As demonstrated in FIG. 4, concentrations of Compound II up to approximately 16 μM had only minimal toxicity (<10% cell kill) to the RECs after the exposures of either 12 or 14 hours, while concentrations up to at least 65 μM resulted in cell kills of less than 25%. These data demonstrate that transient suppression of SK activity does not induce cytotoxicity in the RECs. Therefore, all of the following signaling experiments were conducted with exposures to Compound II of 14 hours or less to ensure the viability of the RECs.

### EXAMPLE 6

Suppression of VEGF- and TNFα-signaling in RECs by SK inhibitors.

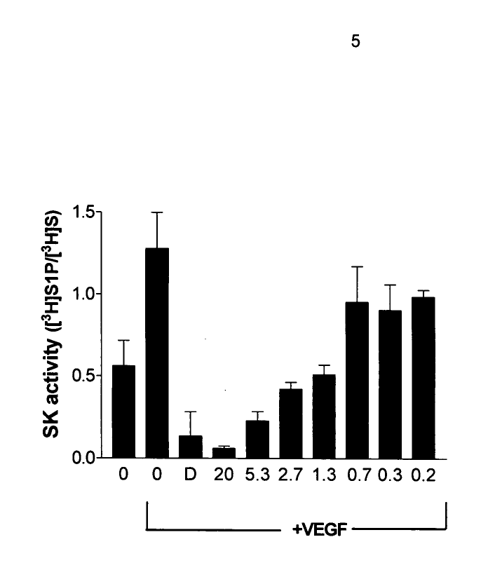

The effects of VEGF on SK activity in bovine RECs using the same cell-based assay as above, except that serum was removed from the cultures 24 hours before the addition of the SK inhibitor and VEGF were tested. This was done to reduce the background level of SK activity responding to growth factors in the serum. As shown in FIG. 5, growth arrest by serum-starvation reduced the basal SK activity, i.e. conversion of [³H]sphingosine to [³H]S1P was lower than in controls shown in FIG. 3. Treatment of the cells with VEGF stimulated SK activity, and this response was dose-dependently inhibited by Compound II, such that the response to VEGF was completely inhibited by concentrations of Compound II of 1.3 μM or higher.

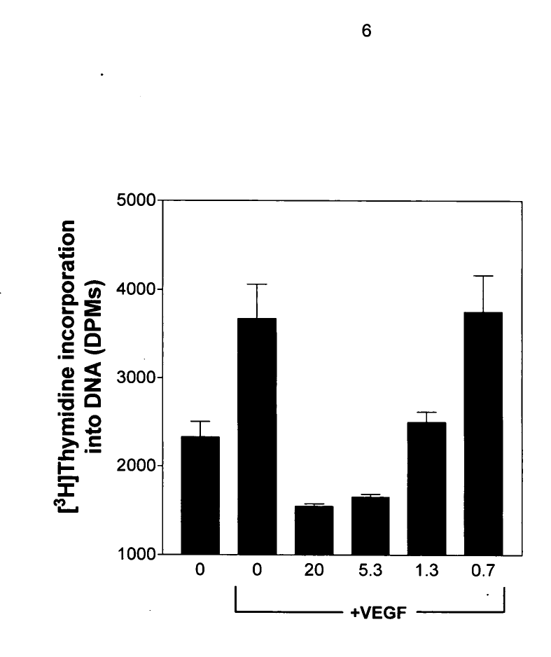

We next evaluated the effects of VEGF on the proliferation of human RECs by measuring the effects of the growth factor of the incorporation of [³H]thymidine into the DNA of serum-starved cells. As shown in FIG. 6, VEGF significantly increased the incorporation of [³H]thymidine into DNA. The effect of VEGF was dose-dependently inhibited by Compound II, such that concentrations of 1.3 μM or higher blocked the mitogenic response to VEGF. Therefore, the inhibition of VEGF-induced SK activity and proliferation are well-correlated in the RECs.

Western analyses were conducted to evaluate the effects of Compound II on signaling proteins known to be regulated in endothelial cells by VEGF or TNFα. In these experiments, human RECs were serum-starved for 24 hours and then exposed to VEGF (50 ng/mL) or TNFα (100 ng/mL) for either 15 minutes or 6 hours. Cell lysates from cells treated with VEGF for 15 minutes were then analyzed for levels of phosphorylated ERK1/2 as a measure of signaling through the Ras-mediated proliferation pathway. Samples from cells treated with TNFα were analyzed for activation of NFκB, i.e. phosphoNFκB, and the down-stream proteins, cyclooxygenase-2 (Cox-2), E-selectin and VCAM-1. Serum-starved human RECs maintained a small level of residual pERK1/2 that was eliminated by treatment of the cells with Compound II. More importantly, VEGF promoted the rapid phosphorylation of ERK1/2, and this response was completely abrogated by treatment of the cells with Compound II. Six hours of exposure to VEGF had no effect on the expression of phosphorylated NFκB, Cox-2, VCAM-1 or E-selectin.

Exposure of the human RECs to TNFα for 6 hours did not affect levels of pNFκB in these cells, and caused a moderate increase in the expression of Cox-2. TNFα caused marked up-regulation of the expression of both E-selectin and VCAM-1, and these responses were completely blocked in cells co-treated with Compound II. Treatment of the cells with an SK inhibitor blocked TNFα-induced prostaglandin E₂ production. The data demonstrate that reduction of S1P by inhibition of SK is an effective means for interfering with proliferative processes induced by VEGF and inflammatory processes induced by TNFα.

### EXAMPLE 7

Suppression of microvessel formation by SK inhibitors.

Since VEGF-mediated angiogenesis and vascular leakage are critical processes in the pathology of diabetic retinopathy, we evaluated the effects of Compound II on the VEGF-induced formation of microvessels by human RECs. The basement membrane-like substrate, Matrigel (Becton-Dickinson, Franklin Lakes, N.J.), was used to induce vessel-like tube formation from human RECs as in previous studies with other cell types (Lee et al., Cancer Lett 208: 89 (2004)). Briefly, 300 μL of Matrigel was pipetted into 24 well plates and allowed to gel at 37° C. for 30 minutes. Human RECs were briefly trypsinized and plated onto the layer of Matrigel at an approximate density of 25,000 cells/cm². VEGF (50 ng/mL in PBS) with either DMSO or an SK inhibitor was added to the media immediately. After 18 hours at 37° C., images were digitally captured with a Retiga Ex camera with bright-field microscopy using a Nikon Eclipse TE300 microscope.

Plating of RECs on Matrigel-coated dishes allowed the cells to migrate to form flat cellular networks. Addition of VEGF to the cultures induced the formation of vessel-like tubes that were more elongated and three-dimensional than the networks in control cultures. Addition of Compound II caused a marked reduction of the formation of networks and tubes in control and VEGF-treated cultures. Isolated cells were commonly visible in the Compound II-treated cultures, whereas single cells were rarely seen in the VEGF-alone treated cultures. Thus, inhibition of SK effectively blocks REC migration and thereby prevents VEGF from promoting the assembly of these cells into microvessels.

### EXAMPLE 8

Maximum tolerated dose of SK inhibitors.

ABC747080 and ABC294640 have been synthesized in amounts sufficient for characterization of their toxicity, pharmacokinetics and in vivo efficacies. The compounds are soluble to at least 15 mg/ml in 50% dimethylsulfoxide:50% phosphate-buffered saline (DMSO:PBS) for intraperitoneal (IP) administration or Polyethylene glycol-400 (PEG400) for oral dosing. Acute toxicity studies using IP dosing demonstrated no immediate or delayed toxicity in female Swiss-Webster mice treated with up to at least 50 mg/kg for ABC747080 or ABC294640. Repeated injections in the same mice every other day over 15 days showed similar lack of toxicity. Each of the compounds could also be administered orally to mice at doses up to at least 100 mg/kg without noticeable toxicity. Therefore, these compounds were suitable for chronic in vivo treatments.

### EXAMPLE 9

Pharmacokinetics of SK inhibitors.

Detailed pharmacokinetic studies were performed on ABC747080 and ABC294640 dissolved in PEG400 or 0.375% Tween-80, respectively. Female Swiss-Webster mice were dosed with 50 mg/kg ABC294640 either intravenously or orally, or 100 mg/kg ABC747080 orally. Mice were anesthetized and blood was removed by cardiac puncture at time points ranging from 1 minute to 8 hours. Concentrations of ABC747080 and ABC294640 were quantified using liquid-liquid extraction and reverse phase HPLC coupled to an ion trap quadrapole mass spectrometer. Control blood samples were spiked with known amounts of internal standard and analyte to identify compound-specific peaks and to develop standard curves for quantification. Pharmacokinetic parameters were calculated using the WINNONLIN analysis software package (Pharsight). Non-compartmental and compartmental models were tested, with the results from the best fitting models shown in Table 2.

TABLE 2
Pharmacokinetic data for SK inhibitors.

| | | Dose | AUC_{0→∞} | AUC_{0→∞} | Tₘₐₓ | Cₘₐₓ | Cₘₐₓ | T_{1/2} |
|---|---|---|---|---|---|---|---|---|
| Compound | Route | (mg/kg) | (μg*h/ml) | (μM*h) | (h) | (μg/ml) | (μM) | (h) |
| ABC294640 | IV | 50 | 56.9 | 137 | 0 | 31.1 | 74 | 1.4 |
| ABC294640 | Oral | 50 | 37.5 | 90.1 | 0.25 | 8 | 19 | 4.5 |
| ABC747080 | Oral | 100 | 475 | 1500 | 1 | 15 | 33 | 32 |

For both compounds, blood levels exceeded the IC₅₀ for inhibition of SK activity during the entire study. ABC747080 demonstrated excellent PK properties, with large Area Under the Curve (AUC) and Cₘₐₓ (maximum concentration reached in the blood) values. ABC294640 demonstrated desirable PK properties as well, with acceptable half life and Cₘₐₓ values. Comparison of oral versus intravenous pharmacokinetics of ABC294640 revealed very good oral bioavailability properties (F=AUC (oral)/AUC (iv)=0.66). These results demonstrate that both ABC747080 and ABC294640 have excellent drug properties, specifically good oral availability with low toxicity.

### EXAMPLE 10

In vivo effects of SK inhibitors on VEGF-induced vascular permeability.

The effects of VEGF on vascular leakage in vivo were measured as described by Miles and Miles (Miles et al., J Physiol 118: 228 (1952)). Groups of female athymic nude mice (approximately 20 g) were given intraperitoneal injections of DMSO alone, Compound 11 (100 mg/kg of body weight) or ABC294640 (75 mg/kg) in a volume of 50 μL. In some experiments, ABC294640 was administered by oral gavage at a dose of 100 mg/kg. After 30 minutes, 100 μL of 0.5% Evan's blue dye in PBS was administered by tail vein injection. Thirty minutes later, mice received the first of 3 sequential (every 30 minutes) intradermal injections of VEGF (400 ng in 20 μL of PBS per injection) on the left hind flank. As a control, similar injections of PBS were administered on the right hind flank. Thirty minutes after the last injection, leakage of the dye from the vasculature into the skin was assessed by measuring the length and width of the spots of blue-colored skin using calipers.

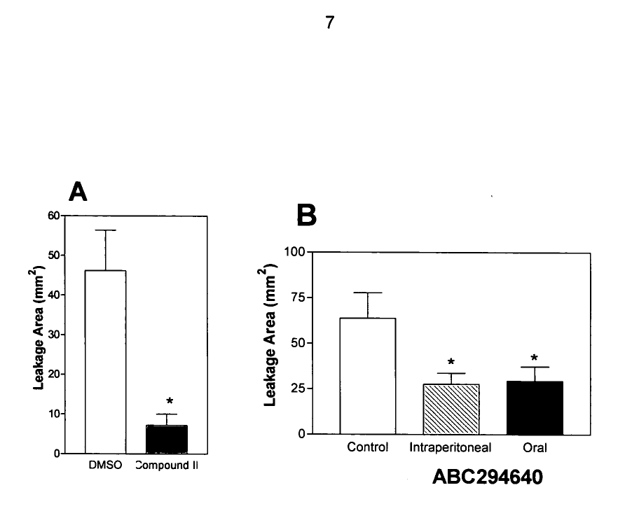

Administration of an intradermal bolus of VEGF results in leakage of the protein-bound dye into the skin indicating a local increase in vascular permeability. When Compound II was administered by intraperitoneal injection one hour before the VEGF treatment, vascular leakage (determined three hours later) was markedly reduced. The extent of vascular leakage was quantified by measuring the blue area, and FIG. 7A demonstrates that Compound II inhibited the in vivo response to VEGF by more than 80%. Similarly, the effects of ABC294640 on VEGF-induced vascular leakage were determined. As indicated in FIG. 7B, either intraperitoneal or oral administration of ABC294640 suppressed the ability of VEGF to promote dye leakage into mouse skin. Therefore, structurally diverse SK inhibitors have a common ability to suppress in vivo vascular leakage in response to VEGF.

### EXAMPLE 11

In vivo effects of SK inhibitors on diabetic retinopathy.

Male Sprague-Dawley rats weighing 150-175 g were used. Diabetes was produced by intraperitoneal injection of streptozotocin (65 mg/kg in citrate buffer) after overnight fasting. Sham-injected non-diabetic animals were also carried as controls. Blood glucose was measured three days post-injection and animals with blood glucose over 250 mg/dL were used as diabetic rats for the study. Blood glucose levels and body weights were monitored weekly throughout the study. On Day 45, retinal vascular permeability was measured in a group of control and diabetic rats (Antonetti et al., Diabetes 47: 1953 (1998), Barber et al., Invest Ophthalmol Vis Sci 46: 2210 (2005)). Briefly, animals were weighed, anesthetized with ketamine/xylazine (80/0.8 mg/kg) and injected with fluorescein isothiocyanate-conjugated bovine serum albumin (FITC-BSA; Sigma catalog number A-9771) into the femoral vein. Following 30 minutes of FITC-BSA circulation, the rats were sacrificed by decapitation. Trunk blood was collected to measure the FITC-BSA concentration, and eyes were quickly enucleated. Each eye was placed in 4% paraformaldehyde for 1 hour and frozen in embedding medium in a bath of isopentane and dry ice. The paraffin-embedded eyes were sectioned on a microtome making 10 μm sections. Sections were dewaxed and viewed with an Olympus OM-2 fluorescence microscope fitted with a Sony CLD video camera. Fluorescence intensities of digital images were measured using Leica Confocal Software (Version 2.61, build 1538, LCS Lite, 2004). The average retinal intensity for each eye was then normalized to non-injected controls analyzed in the same manner and to the plasma fluorescence of the animal. Through serial sectioning of the eye, this technique enables quantification of varied vascular permeability in the retina (Antonetti et al., Diabetes 47: 1953 (1998), Barber et al., Invest Ophthalmol Vis Sci 46: 2210 (2005)).

The remaining control animals were maintained for an additional 6 weeks, i.e. until Day 87, as were the remaining diabetic rats that were divided into untreated, low-dose ABC294640 (25 mg/kg) or high-dose ABC294640 (75 mg/kg) treatment groups. ABC294640 was administered by intraperitoneal injection (dissolved in 0.375% Tween-80) 5 days per week from Day 45 to Day 87. On Day 87, all remaining animals were tested for retinal vascular permeability as described above. Sections were also stained for SK immunoreactivity using the rabbit polyclonal antibodies, and counterstained for nuclei using Hoescht stain.

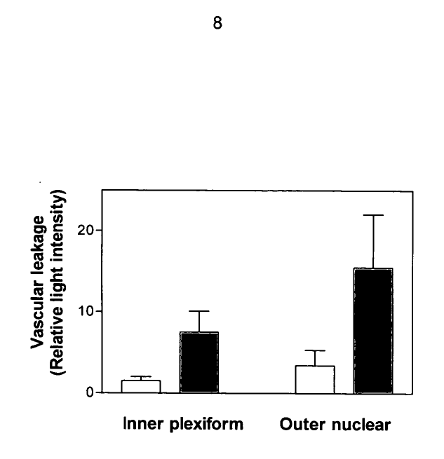

Hyperglycemic rats were left untreated for 45 days to allow the progression of retinopathy. At that time, control and diabetic rats were evaluated for retinal vascular permeability by measuring the leakage of FITC-labeled BSA into the retina using quantitative image analyses. As indicated in FIG. 8, the diabetic animals had substantial increases in the leakage of the labeled BSA into the inner plexiform and outer nuclear layers of the retina. Quantification of the images indicated that there is an approximately 4-fold increase in the amount of FITC-BSA leakage in the retinas from diabetic rats. Therefore, substantial diabetes-induced vascular damage was present before the initiation of treatment with the SK inhibitor.

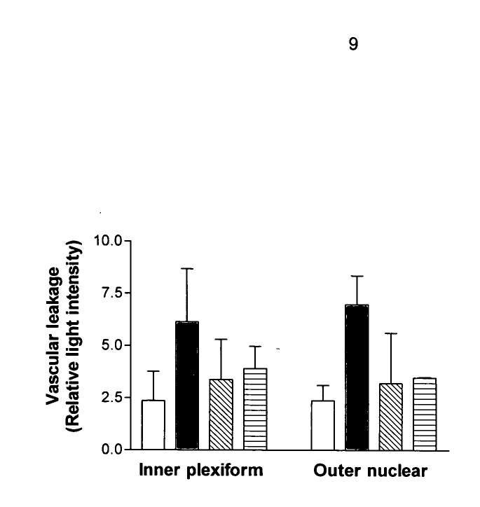

All of the surviving rats were sacrificed on Day 87 and retinopathy was measured as the leakage of FITC-BSA into the retina. As indicated in FIG. 9, retinal vascular permeability in the diabetic rats was significantly elevated compared with the control rats. Diabetic animals that had been treated with the SK inhibitor ABC294640, at either dose, had substantially reduced levels of FITC-BSA leakage than did the untreated diabetic rats. This effect of the compound was manifested in both the inner plexiform layer and the outer nuclear layer of the retina.

Immunohistochemistry with the SK antibody described above was used to evaluate the expression of SK in the retinas of these animals. Fluorescence in the retinal pigment epithelium and the outer segment was non-specific since it was present in samples incubated in the absence of the SK antibody. Retinal sections from control rats had only low levels of specific staining for SK; whereas, SK expression was markedly elevated in the ganglion cell layer and in specific cell bodies and projections at the interface of the inner nuclear layer and the inner plexiform layer. Elevated SK expression was also observed in both the low-dose and the high-dose ABC294640-treated animals. Therefore, the long-term hyperglycemic state appears to be associated with elevation of retinal SK levels that are not normalized by treatment with the SK inhibitor. This expression data indicates that ABC294640 very effectively suppresses SK activity in the diabetic retina, thereby preventing the increased vascular permeability normally present in retinopathy.

### EXAMPLE 12

In vivo effects of SK inhibitors in the Collagen-Induced Arthritis model in mice.

The anti-arthritis activities of the SK inhibitors ABC294640 and ABC747080 were assessed in the Collagen-Induced Arthritis (CIA) model. Female DBA/1 mice were injected subcutaneously in the tail with chicken immunization-grade type II collagen (Chondrex) emulsified in complete Freund's adjuvant (Sigma) at 2 mg/mL. Three weeks later, the mice received a collagen booster in incomplete Freund's adjuvant and were monitored daily thereafter for arthritic symptoms. Once mice reached a threshold paw thickness and clinical score, they were randomized into the following treatment groups: ABC294640 (100 mg/kg given orally each day for 6 days per week), ABC747080 (50 mg/kg given orally each day for 6 days per week) or vehicle (0.375% Tween-80 given under the same schedule). The severity of disease in each animal was quantified by measurement of the hind paw volume with digital calipers. Each paw was scored based upon perceived inflammatory activity, in which each paw receives a score of 0-3 as follows: 0=normal; 1=mild, but definite redness and swelling of the ankle or wrist, or apparent redness and swelling limited to individual digits, regardless of the number of affected digits; 2=moderate redness and swelling of the ankle and wrist and 3=severe redness and swelling of the entire paw including digits, with an overall score ranging from 0-12. Differences among treatment groups were tested using ANOVA.

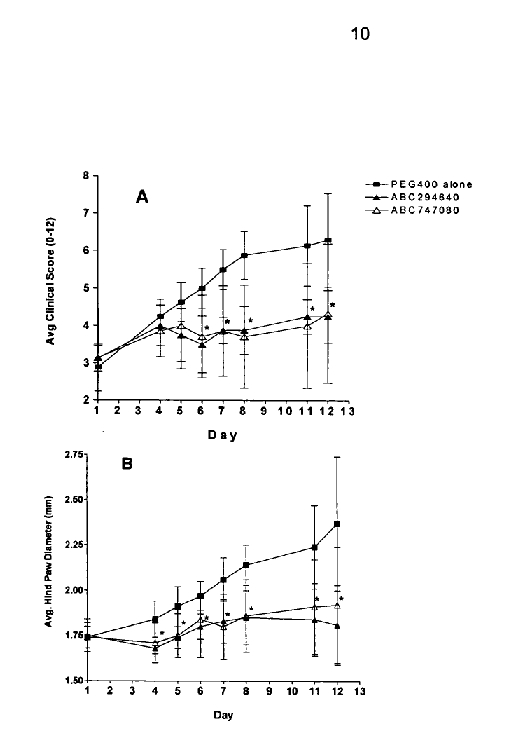

As indicated in FIG. 10, treatment with either SK inhibitor dramatically slowed the inflammation response, measured as either the Average Clinical Score (FIG. 10A) or the Average Hind Paw Diameter (FIG. 10B), with significant decreases beginning at Day 5 of treatment for both endpoints. By the end of the experiment on Day 12, ABC294640 caused a 90% reduction in the increase in hind paw thickness, and a 67% reduction in clinical score compared with vehicle-treated mice. Similarly, ABC747080 caused a 72% reduction in the increase in hind paw thickness, and a 65% reduction in clinical score. Since a 30% reduction in symptoms is considered demonstrative of anti-arthritic activity in this assay, these SK inhibitors surpass the criteria for efficacy in this model.

On Day 12, the mice were euthanized and their hind limbs were removed, stripped of skin and muscle, formalin-fixed, decalcified and paraffin-embedded. The limbs were then sectioned and stained with hematoxylin/eosin. Tibiotarsal joints were evaluated histologically for severity of inflammation and synovial hyperplasia. Collagen-Induced Arthritis resulted in a severe phenotype compared with non-induced mice, manifested as severe inflammation and synovial cell infiltration, as well as significant bone resorption. Mice that had been treated with either ABC294640 or ABC747080 had significantly reduced histologic damage, correlating with the paw thickness and clinical score data.

### EXAMPLE 13

In vivo effects of SK inhibitors in the Adjuvant-Induced Arthritis model in rats.

Adjuvant-induced arthritis is another widely used assay that recapitulates many features of human rheumatoid arthritis, and so is useful in the evaluation of new drug candidates. Age- and weight-matched male Lewis rats (150-170 g) were injected subcutaneously in the tail with 1 mg of Mycobacterium butyricum (Difco, killed dried) suspended in 0.1 ml of light mineral oil. Symptoms of immune reactivity were present after 2 weeks. Responsive rats were randomized into treatment groups, and received oral daily doses (1 ml) of: solvent alone (0.375% Tween-80); 100 mg/kg ABC294640; 35 mg/kg ABC294640; or 5 mg/kg ABC294640, or intraperitoneal injections of indomethacin (5 mg/kg) every other day as a positive control. The severity of disease in each animal was quantified by measurement of the hind paw thickness. As above, a reduction of 30% or greater was considered to be an indication of anti-inflammatory activity in this model.

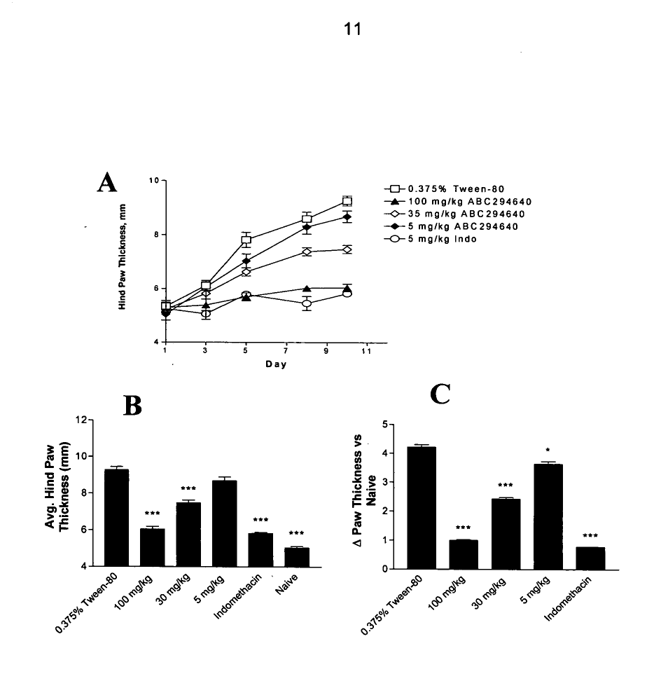

As indicated in FIG. 11, solvent alone-treated rats demonstrated a progressive increase in paw thickness over the course of the next 10 days. ABC294640 inhibited this arthritic response in a dose-dependent manner, with the highest dose having similar therapeutic efficacy as indomethacin. ABC294640 at doses of 5, 35 or 100 mg/kg resulted in 13, 42 and 76 percent reductions in the arthritic response, respectively. Thus, ABC294640 is highly effective against this arthritis model.

### EXAMPLE 14

Antitumor activity of SK inhibitors.

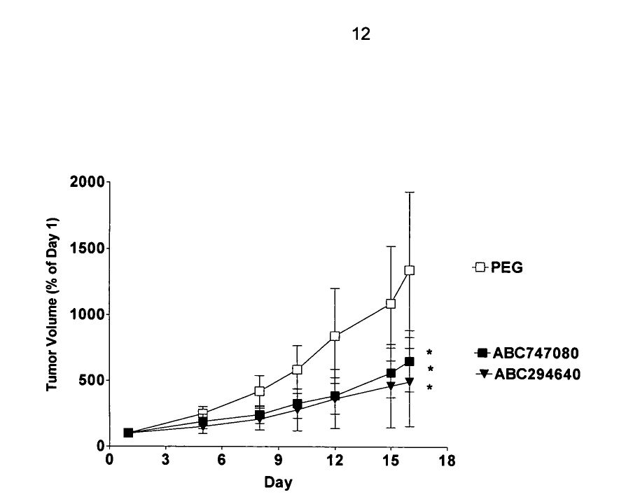

We determined the antitumor activity of representative SK inhibitors in a syngenic mouse tumor model that uses a transformed murine mammary adenocarcinoma cell line (JC, ATCC Number CRL-2116) and Balb/C mice (Charles River) (Lee et al., Oncol Res 14: 49 (2003)). Animals were housed under 12 hour light/dark cycles, with food and water provided ad libitum. Tumor cells (1×10⁶) were implanted subcutaneously, and tumor volume was calculated using the equation: (L×W²)/2. Upon detection of tumors, mice were randomized into treatment groups. ABC747080 or ABC294640 was dissolved in PEG400 and orally administered to fasted mice on odd days at a dose of 100 mg/kg. As indicated in FIG. 12, both compounds had antitumor activity without toxicity to the mice. ABC747080 and ABC294640 inhibited tumor growth by 56 and 69%, respectively. The body weights of SK inhibitor-treated groups were not different from those of the control group.

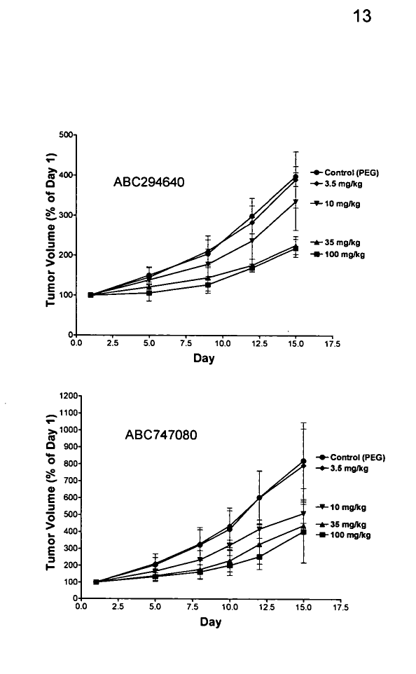

Dose-response studies of the in vivo activities of ABC747080 and ABC294640 have also been conducted. As demonstrated in FIG. 13, both compounds cause dose-dependent inhibition of tumor growth by orally administered ABC747080 with an EC₅₀ of 10 mg/kg, and of ABC294640 with an EC₅₀ of approximately 35 mg/kg, consistent with their respective AUCs. Comparison with the potencies in the tumor studies with the toxicity data described above reveals that ABC294640 has a therapeutic index of greater than 30 (1000 mg/kg nontoxic dose/35 mg/kg antitumor activity) and ABC747080 has an index greater than 40 (400 mg/kg nontoxic dose/10 mg/kg antitumor activity). Thus, these SK inhibitors have excellent therapeutic windows.

We claim:

1. A method for treating an angiogenic disease comprising delivering to a patient a compound or pharmaceutical composition in an amount effective to inhibit sphingosine kinase activity.

2. A method of claim 1 wherein said angiogenic disease is selected from the group consisting of ocular angiogenic disease, arthritis, cancer, psoriasis, Kaposi's sarcoma, hemangiomas, myocardial angiogenesis, and atherosclerosis.

3. A method of claim 2 wherein said arthritis is selected from the group consisting of rheumatoid arthritis, osteoarthritis, Caplan's Syndrome, Felty's Syndrome, Sjogren's Syndrome, ankylosing spondylitis, Still's Disease, Chondrocalcinosis, gout, rheumatic fever, Reiter's Disease and Wissler's Syndrome.

4. A method of claim 2 wherein said ocular angiogenic disease is selected from the group consisting of diabetic retinopathy, choroidal neovascularization, retinopathy of prematurity (retrolental fibroplasias), macular degeneration, corneal graft rejection, rubeosis, neuroscular glacoma and Oster Webber syndrome.

5. A method of claim 2 wherein said cancer is selected from the group consisting of solid tumors, hematopoietic cancers and tumor metastases.

6. A method of claim 5 wherein said solid tumor is selected from the group consisting of head and neck cancers, lung cancers, gastrointestinal tract cancers, breast cancers, gynecologic cancers, testicular cancers, urinary tract cancers, neurological cancers, endocrine cancers, skin cancers, sarcomas, mediastinal cancers, retroperitoneal cancers, cardiovascular cancers, mastocytosis, carcinosarcomas, cylindroma, dental cancers, esthesioneuroblastoma, urachal cancer, Merkel cell carcinoma and paragangliomas.

7. A method of claim 5 wherein said hematopoietic cancer is selected from the group consisting of Hodgkin lymphoma, non-Hodgkin lymphoma, chronic leukemias, acute leukemias, myeloproliferative cancers, plasma cell dyscrasias, and myelodysplastic syndromes.
